(19) United States
(12) Patent Application Publication
(10) Pub. No.: US 2010/0130622 A1
(43) Pub. Date: May 27, 2010

(54) PHARMACEUTICAL PREPARATION FOR PREVENTING AND/OR TREATING PERIODONTAL DISEASE, AGENT FOR ORAL HYGIENE COMPRISING THEREOF, AND FOOD COMPRISING THEREOF

(75) Inventors: Je-Tae WOO, Tokyo (JP); Masato Ohta, Chiba (JP); Takayuki Yonezawa, Aichi (JP); Toshiaki Teruya, Kanagawa (JP); Byung-Yoon Cha, Aichi (JP); Kazuo Nagai, Tokyo (JP)

Correspondence Address: KRATZ, QUINTOS & HANSON, LLP, 1420 K Street, N.W., Suite 400, WASHINGTON, DC, 20005, US

(73) Assignee: Kazuo Nagai, Tokyo (JP)

(21) Appl. No.: 12/275,643

(22) Filed: Nov. 21, 2008

(51) Int. Cl. A61K 31/05

(52) U.S. Cl. 514/733

(57) ABSTRACT

The present invention provides a pharmaceutical composition for preventing and/or treating periodontal disease comprising at least one having dental root-periodontal tissue formation promotion effect selected from the group consisting of a compound shown in a following formula (I).

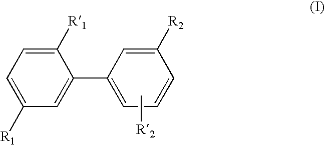

(In the formula, R₁ and R₂ are independently functional groups selected from a group consisting of a substituted or non-substituted group of a linear alkyl group, a branched alkyl group, a linear alkenyl group and a branched alkenyl group with having the carbon number of 3 to 5. R′₁ and R′₂ are independently a hydrogen atom, hydroxyl group, or alkoxy group having the carbon number of 1 to 3.)

### TECHNICAL FIELD

The present invention relates to an application way of compounds among biphenyl compounds derived from crude drug components shown in chemical formula (I) described in below. More specifically, the present invention relates to pharmaceutical compositions for preventing and/or treating periodontal disease for human and animals, oral cavity hygiene agents, and composition for food comprising at least one of the biphenyl compounds shown in chemical formula (II) or (III) described in below, pharmaceutically acceptable salts thereof, or hydrates thereof.

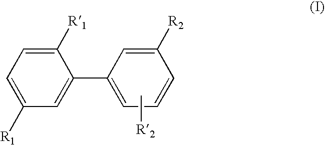

(In the formula, R₁ and R₂ are independently functional groups selected from a group consisting of substituted or non-substituted linear alkyl group, branched alkyl group, linear alkenyl group and branched alkenyl group with having the carbon number of 3 to 5. R′₁ and R′₂ are independently a hydrogen atom, hydroxyl group, or alkoxy group having the carbon number of 1 to 3.)

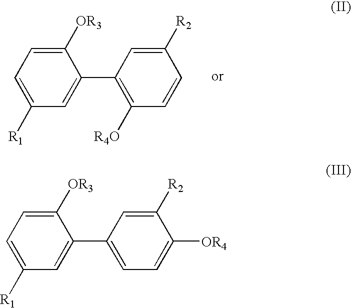

(In the formula, R₁ and R₂ are independently functional groups selected from a group consisting of a substituted or non-substituted linear alkyl group, branched alkyl group, linear alkenyl group and branched alkenyl group with having the carbon number of 3 to 5. R′₁ and R′₂ are independently a hydrogen atom, hydroxyl group, or alkyl group having the carbon number of 1 to 3.)

### BACKGROUND OF THE INVENTION

Periodontal disease is almost national affliction, because more than 80% of Japanese people over 30 years old affected with it. However, patients do not feel pain unless the symptom is worsening to become severe condition. Therefore, it is seen as the disease being characterized to cause serious disease easily. In advanced periodontal disease, discomfort or pain derived from mastication of food causes anorexia and a breath accompanying bad smell. As a result, it may bring insufficient nutrition or communication disorder. In such a case, specialized medical treatment by a dentist is performed in the patient, but it is difficult to halt the development to severe symptom and the patient loses their teeth. On the other hand, it is known that healthy aged people have enough number of residual teeth.

In recent years, periodontal disease is spreading in not only human, but also animal companions such as cats and dog fed softened food in a house, and this is viewed in suspicion.

In the patient of periodontal disease, cement, periodental membrane and alveolar bone, these compose tissue, are deteriorated. Accordingly, the treatment of the periodontal disease is performed by removing causative agent that causes the inflammation such as tartar or dead cement to facilitating cure of the tissues in human. However, it is difficult to re-build new periodental tissues after the tissues already have been deteriorated.

### DISCLOSURE OF THE INVENTION

Problems to be solved by the Invention

There are further needs for the treatment of periodontal disease to halt to develop the severe symptom to avoid loosing teeth, and maintain good nutrition state, performing a communication with no difficulty at any age when periodontal disease is treated. Particularly, it is important for the aged people to have a diet by masticating with their own teeth to prevent to reduce the Quality of Life (hereinafter, it is referred to as “QOL”) and bone disease. Furthermore, there is no way except extracting the teeth for the patient animal having periodental disease other than human, and it reduces the QOL of the animal companions markedly.

In order to solve such problems and maintain their teeth stably, it is expected to find out substances having the prophylactic effects to periodental disease and develop agents in a practical level.

On the other hand, the treatment agent should be essentially administrated directly into oral cavity. Therefore, it is necessary the treatment agent to have the high curative effects and safety as the same as that for the bone disease treatment.

In order to treat the periodontal disease, surgical operations are employed. In GTR method that uses non-absorptive membrane to be embedded, the membrane should be taken out from the embedded site later.

Alternatively, EMDOGAIN (Registered trademark) is used, it sometimes causes anaphylaxis such as urticaria and itching skin reaction, local inflammation, angular cheilitis, and so forth are reported as a concurrent disease after the surgical operation.

Accordingly, there is high social need to develop a treatment method that does not use the surgical operation as far as possible, and use the material which seldom causes the anaphylaxis.

### Means for Solving the Problem

The present invention is completed under the above-mentioned situation.

Namely, the present invention is completed under the situations in below. Inventors of the present invention proceed to screen compounds having dental root-periodontal tissue formation activity contained in plants used as the crude drugs or the herb teas from ancient age, considering for showing high pharmacological effect and safety.

The inventors of the present invention screened compounds having dental root-periodontal tissue formation activity mainly among the components contained in plants used as the crude drugs or the herb teas from ancient age, considering for showing high pharmacological effect and safety. As a result, the inventors found out that the known compounds have conventionally unknown and novel activities to promote dental root-periodontal tissue formation.

There are mentioned, for example, honokiol, magnolol and the like as biphenyl compounds having such activities. It is known that these compounds have effects for OH radical activity inhibition and lipoxidation suppression (see Patent reference 1), that ethanol extracts from Magnolia Cortex has the effect for acid phosphatase activity suppression (see Patent reference 2), and that diluted ethanol extracts or hot water extracts of bark, root bark, trunk material, and leaves of Japanese big-leaf magnolia (Honoki) have the effect for collagenase activity suppression (see Patent reference 3).

However, there are no reports that above-mentioned biphenyl compounds have dental root-periodontal tissue formation promotion activities. Furthermore, since these compounds have been used long time in Japan or China as the crude drugs containing them, it is demonstrated that they are highly safe.

[Patent reference No. 1] JP H11-209276

[Patent reference No. 2:] JP H10-338631

[Patent reference No. 3:] JP H05-51316

The present invention is completed under the above-mentioned situation by using dental root-periodontal tissue formation promotion activities.

Namely, the present invention is a pharmaceutical composition for preventing and/or treating periodontal disease comprising at least one selected from the group consisting of a compound shown in the following formula (I), a pharmaceutically acceptable salts thereof, and the hydrates thereof.

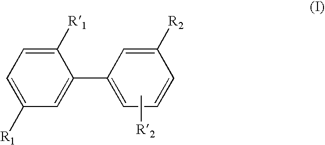

In the formula, R₁ and R₂ are independently functional groups selected from a group consisting of a substituted or non-substituted linear alkyl group, branched alkyl group, linear alkenyl group and branched alkenyl group with having the carbon number of 3 to 5. R′₁ and R′₂ are independently a hydrogen atom, hydroxyl group, or alkoxy group having the carbon number of 1 to 3.

Wherein, the compound shown in the above-mentioned formula (I) is preferably that shown in the following formulae (II) or (III).

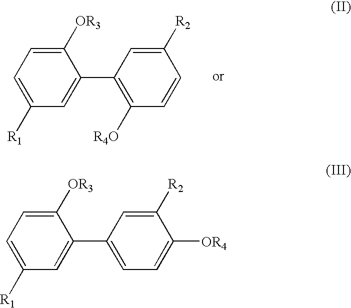

In the formula, R₁ and R₂ are independently functional groups selected from a group consisting of a substituted or non-substituted linear alkyl group, branched alkyl group, linear alkenyl group and branched alkenyl group with having the carbon number of 3 to 5. R₃ and R₄ are independently a hydrogen atom, hydroxyl group, or alkoxy group having the carbon number of 1 to 3.

Wherein, the compound shown in the above-mentioned (II) or (III) are preferably those shown in the following formulae (IV) or (V), the physiologically acceptable salts thereof, or the physiologically acceptable hydrates thereof, which has the dental root-periodontal tissue formation promotion effect.

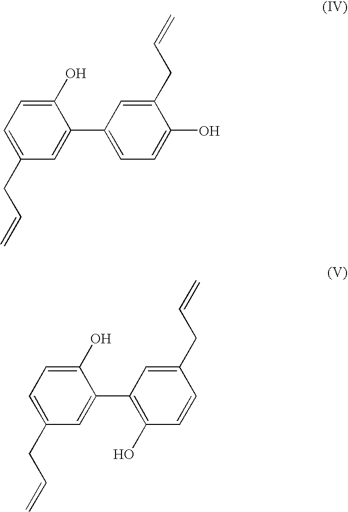

Furthermore, when the composition having the dental root-periodontal tissue formation effect is prepared by using at least two of the above-mentioned compound, or the physiologically acceptable salts thereof or the hydrates thereof, it preferably comprises one of the compounds shown in the above-mentioned formulae (I) to (V), or the physiologically acceptable salts thereof or the hydrates thereof.

The pharmaceutical composition, the present invention, preferably contains one selected from the group consisting of the compounds shown in the formulae (I) to (V), the physiologically acceptable thereof, or the pharmaceutically acceptable hydrates thereof, extracted from the bark of high tree belonged to Magnoliaceae, having the dental root-periodontal tissue formation effect.

Here, the high trees belonged to the above-mentioned Magnolia (Magnoliaceae) is preferably selected from the group consisting of Magnolia officinalis (Karahou in Japanese name), Magnolia officinalis var. biloba (Oubakouboku in Japanese name), Magnolia hypoleuca (Japanese big-leaf magnolia), Magnolia macrophylla, Magnolia obovata (Hounoki, Japanese big-leaf magnolia in Japanese name), Magnolia salicifola (Willow-leaved magnolia), Magnolia stellata (Star magnolia), Magnolia virginiana (Himetaisanboku in Japanese name, Sweet bay), Magnolia delavayi, Magnolia kobus, Magnolia sieboldii (Oobayamarenge in Japanese name), and Magnolia wilsonii, because they contain the above-mentioned compounds.

Among these high trees belonged to Magnolia (Magnoliaceae), the tree is more preferably selected from the group consisting of Magnolia officinalis, Magnolia officinalis var. biloba, Magnolia hypoleuca, Magnolia obovata, Magnolia salicifola, Magnolia stellata, Magnolia virginiana, and Magnolia sieboldii, from the viewpoint of the contained amounts of the above-mentioned compounds.

The pharmaceutical composition having maintenance effect of dental root-periodontal tissue or promotion effect of dental root-periodontal tissue formation is prepared by adding the above-mentioned compounds, salts thereof or extracts from the plants to tooth powder or tooth paste, gargle and the like in an suitable amount.

Present invention is also a pharmaceutical preparation for preventing and/or treating periodontal disease containing one that has dental root-periodontal tissue formation promotion effect, selected from the group consisting of the above-mentioned compounds, physiologically acceptable salts thereof, and hydrates thereof.

The pharmaceutical preparation preferably contains one selected from the group consisting of the compound shown in the chemical formulae (I) to (V) having the dental root-periodontal tissue formation effect in the extracts from the plant selected from the group consisting of Magnolia officinalis, Magnolia officinalis var. biloba, Magnolia hypoleuca, Magnolia macrophylla, Magnolia obovata, Magnolia salicifola, Magnolia stellata, Magnolia virginiana, Magnolia delavayi, Magnolia kobus, Magnolia sieboldii, and Magnolia wilsonii, physiologically acceptable salts thereof, and hydrates thereof as the active ingredient.

Furthermore, the present invention is a functional food comprising one selected from the group consisting of the compound shown in the chemical formulae (I) to (V) having dental root-periodontal tissue formation effect, physiologically acceptable salts thereof, and hydrates thereof as the active ingredient.

As added substances for the functional food, one selected from the group consisting of the physiologically acceptable salts thereof and the hydrates thereof as the active ingredient, and sodium salt, potassium salt, ammonium salt, magnesium salt thereof, monohydrates and dihydrates thereof are more preferable.

Concretely, one selected from the group consisting of the compounds shown in the following formulae (IV) and (V), the physiologically acceptable salts thereof and the hydrates thereof are more preferable. More preferably, it is selected from the group consisting of sodium salt, potassium salt, ammonium salt, magnesium salt thereof, monohydrates and dihydrates thereof are more preferable.

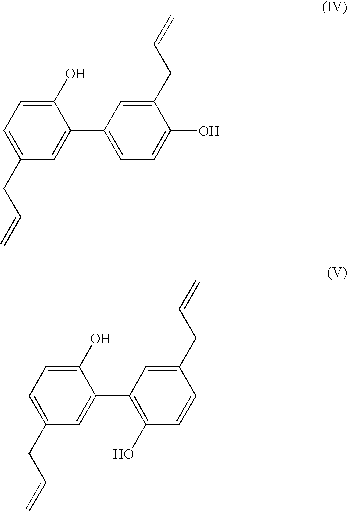

Furthermore, the present invention is preferably a functional food comprising an extract that is extracted from a bark or a branch bark of high trees belonged to Magnoliaceae as mentioned above; wherein the extract contains one selected from the group consisting of the compounds shown in the above formulae (I) to (V), physiologically acceptable salts thereof, and physiologically acceptable hydrates thereof. Intake of the functional foods containing these compounds and the like enables to prevent periodontal disease. Here, the high trees belonged to the Magnolia (Magnoliaceae) are the same as described above.

According to the present invention, the pharmaceutical composition, the pharmaceutical preparation, the functional foods, and the healthy foods, having excellent dental root-periodontal tissue formation promotion effect, for preventing and/or treating periodontal disease are provided.

### BRIEF DESCRIPTION OF THE DRAWINGS

FIG. 1A is a side view showing promotion effects of the dental root extension and periodontal tissue formation, when a reference sample using D-PBS only was embedded into the dental pulp;

FIG. 1B is a sectional view showing promotion effects of the dental root extension and periodontal tissue formation, when a reference sample using D-PBS only was embedded into the dental pulp;

FIG. 1C is the side view showing promotion effects of the dental root extension and periodontal tissue formation, when an agarose bead impregnated with honokiol was embedded into the dental pulp; and

FIG. 1D is the sectional view showing promotion effects of the dental root extension and periodontal tissue formation, when the agarose bead impregnated with honokiol was embedded into the dental pulp.

### BEST MODE FOR CARRYING OUT THE INVENTION

The first embodiment of the present invention is a pharmaceutical composition for preventing and/or treating periodontal disease comprising at least one selected from the group consisting of the compound shown in the following formula (I), the physiologically acceptable salt thereof, and the hydrate thereof, having the promotion effect for the dental root-periodontal tissue formation, as an active ingredient.

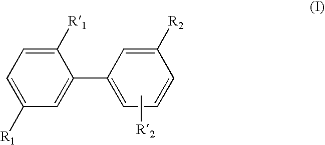

In the formula, R₁ and R₂ are independently functional groups selected from a group consisting of a substituted or non-substituted linear alkyl group, branched alkyl group, linear alkenyl group and branched alkenyl group having the carbon number of 3 to 5. R′₁ and R′₂ are independently a hydrogen atom, hydroxyl group, or alkoxy group having the carbon number of 1 to 3.

Here, the compound shown in the above formula (I) is preferably either one of those shown in the following formulae (II) or (III).

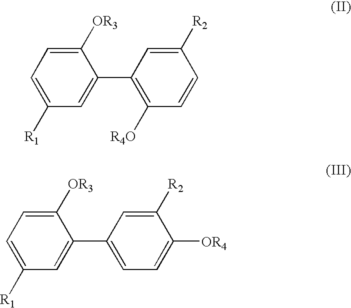

In the formula, R₁ and R₂ independently show the linear or the branched alkyl group or alkenyl group, those having the carbon number of 3 to 5. These functional groups may be substituted or non-substituted. R₃ and R₄ are preferably independently a hydrogen atom, hydroxyl group, or alkoxy group having the carbon number of 1 to 3.

Here, the compound shown in the above-formulae (II) or (III) are preferably one of the compounds shown in the following formulae (IV) or (V), physiologically acceptable salts thereof, or physiologically acceptable hydrates thereof, having the dental root-periodontal tissue formation promotion effect.

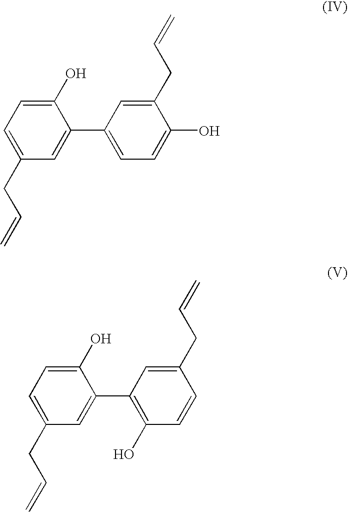

The above-described compounds, the composition having the dental root-periodontal tissue formation promotion effect may be prepared by using the combination of the physiologically acceptable salts thereof or hydrate thereof. In this case, it preferably contains the compound shown in the above-mentioned formulae (I) to (V), the physiologically acceptable salts thereof or the physiologically acceptable hydrate thereof.

The above-described pharmaceutical composition, also the present invention, is more preferably the extract, containing one among the compound shown in the above-mentioned formulae (I) to (V), the physiologically acceptable salts thereof, and the physiologically acceptable hydrates thereof, and the extract is obtained from the bark or branch bark of the high tree belonged to Magnoliaceae, having the dental root-periodontal tissue formation promotion effect.

Among these plants, as the high tree belonged to Magnolia (Magnoliaceae) as described above, there can be mentioned, for example, Magnolia officinalis, Magnolia officinalis var. biloba, Magnolia hypoleuca, Magnolia macrophylla, Magnolia obovata, Magnolia salicifola, Magnolia stellata, Magnolia virginiana, Magnolia delavayi, Magnolia kobus, Magnolia sieboldii, and Magnolia wilsonii. Since these contain the compounds as described above, they are preferably used.

Among the high trees belonged to Magnolia (Magnoliaceae), one selected from the group consisting of Magnolia officinalis, Magnolia officinalis var biloba, Magnolia hypoleuca, Magnolia obovata, Magnolia salicifola, Magnolia stellata, Magnolia virginiana, and Magnolia sieboldii has an advantage that the content amounts of the compound described above is high. Therefore, they are more preferably used.

The compound as described above, the salt thereof, or the extract therefrom may be added to tooth paste, gargle and the like in a suitable amount.

The second embodiment of the present invention is also the pharmaceutical preparation for preventing and/or treating periodontal disease comprising the above-mentioned compound, the physiologically acceptable salts thereof and the hydrates thereof as described above, those have the dental root-periodontal tissue formation promotion effect, as the active ingredient.

The pharmaceutical preparation preferably comprises the extracts containing ones selected from the group consisting of the compound shown in the above formulae (I) to (V), the physiologically acceptable salts thereof, and the hydrates thereof, as the active ingredient. As the raw materials for the extract, there can be mentioned, for example, Magnolia officinalis, Magnolia officinalis var. biloba, Magnolia hypoleuca, Magnolia macrophylla, Magnolia obovata, Magnolia salicifola, Magnolia stellata, Magnolia virginiana, Magnolia delavayi, Magnolia kobus, Magnolia sieboldii, and Magnolia wilsonii. Extraction methods for the extract from these are described in later.

Here, the pharmaceutical preparation is preferably a dosage form that can be administrated per os or that can be directly administrated to the affected area, in view of the treatment efficiency. When the dosage form can be administrated per os is formulated, it is preferably formulated as one selected from tablets, powders, capsules, granules, pills, troches, and liquids. When the dosage form can be directly administrated to the affected area is formulated, it is preferably formulated as one selected from microcapsules, gels, and liposome. As an external preparation, there are mentioned, for example, ointment, creams, cataplasm, sprays, injectables and the like, and also the membrane (sheet) formulation as the same as those used in GTR method described above can be mentioned.

These compositions for preventing and/or treating periodontal disease may be applied to both of humans and animals.

The third embodiment of the present invention is the functional food comprising one selected from the group consisting of the compounds shown in the above formulae (I) to (V), the physiologically acceptable salts thereof, and the hydrates thereof. The functional food has the dental root-periodontal tissue formation promotion effect.

When adding these to the functional foods, those selected from the group consisting of the physiologically acceptable salts thereof and the hydrates thereof may be preferably used; for example, there can be mentioned sodium salts, potassium salts, magnesium salts thereof, mono-hydrates, dihydrates thereof and the like.

Concretely, the compound shown in the following formulae (IV) and (V), the physiologically acceptable salts thereof, hydrates thereof, and the like are more preferably used. When sodium salts, potassium salts, magnesium salts thereof, mono-hydrates or dihydrates thereof are used, there are large merits in the production of the functional food.

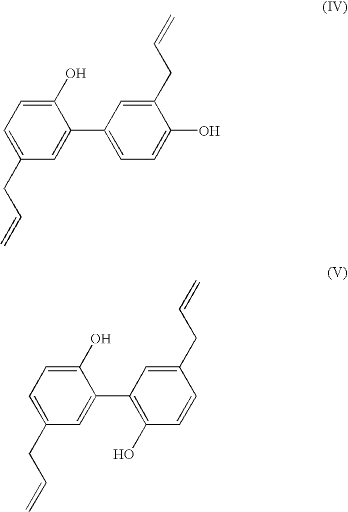

The functional food also contains the extract obtained from the bark or branch bark of the high tree belonged to Magnolia, and the extract preferably contains one among the compounds shown in the above-mentioned formulae (I) to (V), the physiologically acceptable salts thereof, and the physiologically acceptable hydrates thereof. Taking the functional food containing the compounds and the like enables to prevent the periodontal disease. Wherein, the high tree belonged to Magnolia (Magnoliaceae) is the same as those described above.

The functional food of the present invention may be used to aid for preventing and/or treating the periodontal disease. When the functional food is used for such applications, the content of these in the food is preferably 0.1 to 5 mg per 100 mg, more preferably 0.3 to 1 mg. When the content is in the range between 0.3 to 1 mg, it gives the highest efficiency for supplementing the prevention and/or treatment of the periodontal disease.

Addition of the above-mentioned compounds or extracts from the plant to bread, cookies, biscuits and the like, non-alcohol beverages such as tea, coffee and cocoa, soft drinks, and fruits juice, confectionaries such as candies (drops), chocolates, and the like, chewing gum and the like enable to give periodontal tissue preventing effect to them.

Also, the composition of the present invention described above may be used solely, or in combination of two or more. According to the conventional method, these compositions may be formed to the powders, granules, tablets, capsules to form healthy food having dental root-periodontal tissue formation promotion effect.

Furthermore, addition of them to beverages or feed for the animal companions pertain them periodontal disease prevention effects. Here, the method for preparing powder of the above described extracts or compounds is as mentioned above.

The biphenyl compounds such as honokiol as shown in the formula (IV) and magnolol as shown in the formula (V) may be manufactured by using the known method or according to it to be obtained, or purchased commercially available one to be used.

The biphenyl compound as described above is obtained by being extracted from the plant sources and then isolated. As the plant sources, there are mentioned crude drugs; in concrete, there are mentioned, for example, Karakouboku or Wakouboku obtained from Magnolia officinalis, Magnolia officinalis var. biloba, Magnolia hypoleuca, Magnolia obovata from Japan or China. One example for isolating honokiol by using the crude drug as the plant source is shown in below.

Extracts are obtained by adding hydrous methanol or hydrous ethanol to chopped bark and branch bark of Magnolia obotava belonged to Magnoliaceae. The obtained extracts are dissolved into water, and then degreased by using low polar solvent such as petroleum ether, benzene and so forth. Subsequently, it is subjected to butanol extraction, and honokiol and its derivatives are extracted into butanol phase. Here, honokiol and its derivatives include pharmaceutically acceptable salts of honokiol or hydrates thereof.

Honokiol and its derivatives extracted as described above may be used as crude extracts as is, and used to obtain purified samples by purification depending on the needs. The purification may be performed by using the conventional method. For example, by subjecting the extract to a step gradient column chromatography, honokiol and its derivatives are eluted separately to be obtained purified components, for example, as white powders and so forth; in the step gradient column chromatography, silica gels are used as a steady phase, and chloroform/methanol is used as a mobile phase, increasing methanol contents sequentially.

Alternatively, crystalline of honokiol obtained as described above is treated by using the conventional method, and then mixed with the excipient and so forth described in later to obtain the pharmaceutical composition.

Magnolol and other compounds shown in the above formulae (I) to (III), the physiologically acceptable salts thereof, or the hydrates thereof may be extracted and purified similarly to the case of honokiol.

The compounds, the physiologically acceptable salts thereof, or the hydrates thereof obtained as described above may be used to prepare the pharmaceutical composition as described in below.

As the pharmaceutical preparation comprising the compound as the active ingredient, there are mentioned dosage from for the oral administration and others to be administrated directly to an affected area. As the oral administration dosage form, there are mentioned such as tablets, powders, capsules, pills, troches, and variety of liquids. The above-mentioned tablets contain sugarcoated tablets, coated tablets, buccal preparations and the like, and the capsules contain both of hard and soft capsule preparations. The powder preparations also contain coated powder preparations. The liquid preparations contain suspension, emulsion, syrups, elixir, and the syrup contains also dry syrup.

As the dosage form to be directly administrated to the affected site, there are mentioned such as gel preparations and microcapsule preparations, liposome preparations and the like.

Note that each preparation includes not only non-sustained preparations, but also sustained preparations.

These preparations may be formulated according to the known pharmaceutical preparation method by using pharmacologically acceptable carrier, excipient, disintegrator, lubricant, colorant, and so forth, for formulating the preparation, described on Japanese Pharmacopoeia, when they are formulated.

As these carriers or excipients, for example, there are mentioned such as lactose, glucose, sucrose, mannitol, potato starch, corn starch, calcium carbonate, calcium phosphate, calcium sulfate, crystalline cellulose, powdered glycyrrhiza extract, powdered gentian, and so forth.

As a binder, for example, there are mentioned such as the starch, tragacanth gum, gelatin, syrup, polyvinyl alcohol, polyvinylether, polyvinylpyrrolidone, hydroxypropylcellulose, methylcellulose, ethylcellulose, carboxymethylcellulose, and so forth.

As the disintegrator, for example, there are mentioned such as starch, agar, powdered gelatin, sodium carboxymethylcellulose, calcium carboxymethylcellulose, crystalline cellulose, calcium carbonate, sodium bicarbonate, sodium alginate and so forth; as the lubricant, for example, there are mentioned such as magnesium stearate, talc, hydrogenated vegetable oil, macrogol and so forth.

The colorant, which is acceptable to be added to the pharmaceutical preparation, can be used with no limitation. Except these additives, a corrigent and so forth cam be used depending on the necessity.

In order to prepare the composition of the present invention to powder from, the extract obtained in the production process is concentrated, and dried by using the method such as freeze-drying, spray-drying, and vacuum-drying; then the dry solid may be ground by using a sample mill, blender, mixer and the like. Furthermore, depending on the needs, corn starch, potato starch, dextrin, oystershell powders and the like may be added.

Optionally by adding the above-mentioned binder and the like to the powders obtained as described above, and compressing, the tablets may be prepared. After preparing the tablet, it is coated by using coating agent such as sucrose, gelatin and so forth to prepare sugarcoated tablet. By using other coating agent, enteric coating preparation may be also prepared.

Furthermore, the powder is used to prepare granulated powders to prepare the granules formulation. The powder or the granule is packed into the capsules made of ethylcellulose, gelatin and the like in suitable amounts to prepare the capsule.

When the tablet or the granule is prepared, as necessary, it may be coated by using sucrose, gelatin, hydroxypropylcellulose, purified shellac, gelatin, glycerin, sorbitol, ethylcellulose, hydroxypropylcellulose, hydroxypropylmethylcellulose, polyvinylpyrrolidone, cellulose acetate phthalate, hydroxypropylmethylcellulose phthalate, methylmethacrylate, methacrylate polymer and the like as a single coating or plural coatings.

When the liquid preparation is prepared by using the above-mentioned compounds, physiologically acceptable salts thereof and the hydrate thereof, as necessary, pH adjusters, buffering agents, stabilizers, solubilizing agents and the like may be added.

According to the formulation performed by combining the composition of the present invention with the carrier, the excipient, the binder and the like, as necessary, the pharmaceutical preparation for preventing and/or treating periodontal disease are provided.

It is explained by exemplifying to produce gel preparation, agarose beads, including honokiol.

Firstly, honokiol is diluted so as to be predetermined concentration by using the desirable buffer solution to prepare honokiol solution. The agarose beads are washed by using phosphate-buffered saline and the honokiol solution is added to the beads in the predetermined volume, and then incubated them in the predetermined time and temperature. The beads adsorbing honokiol is thus prepared the gel preparation.

Wherein, the concentration of honokiol may be about 1 to 30 μM. As desired buffers, Dulbecco's-modified phosphate-buffered saline (D-PBS, Dainippon Pharmaceuticals Inc.) and so forth may be used, and as the agarose beads, Affi-gel agarose beads (Bio-Rad Laboratories, Inc.) may be used. Furthermore, incubation time and the temperature may be for 30 to 75 minutes, and in the range of 20 to 37° C.

When the promoting agent for forming dental root-periodontal tissue is administered to the patients or patient animals, the dosage may be depending on the conditions such as their thickness of the symptom, age, weight, and of the affected area and so forth. When the gel preparation as described above is prepared, it has an advantage to show high promotion effect for forming dental root-periodontal tissue by being directly embedded to the affected area.

In general, the agent is administrated for an adult in the range between about 1 mg/kg to about 2,000 mg/kg per day, preferably about 1 mg/day to about 1,000 mg/day per os or in parenteral, once or more than once a day. Depending on the conditions as described above, the number of doses a day and the amount may be increase or decrease optionally.

When the pharmaceutical preparation comprises the pharmaceutical preparation including honokiol solely or in combination with other biphenyl compounds, for example, magnolol, as the active ingredients, the honokiol content in the preparation is preferably in the range between 0.1 to 100 mg, more preferably 0.1 to 50 mg, further more preferably 0.3 to 10 mg.

When the amounts of honokiol and other biphenyl compounds as the active ingredients contained in the agent is less than the lower limit, it can not sufficiently show the promoting effect for forming dental root-periodontal tissue. In contrast, when they are added to the agent more than the upper limit, the effect should be derived from the added amounts is not shown, but also the cytotoxicity to cause undesirable side for the body may be shown.

The above-mentioned compositions are added to, for example, breads, cookies, biscuits, wheat to be supplemented to rice and cereals, noodles such as Japanese wheat noodles, buckwheat noodles, pasta and others, dairy food such as cheese, yogurt and others, jam, mayonnaise, soy bean product such as soy bean paste, soy source and others, nonalcoholic beverage such as tea, coffee and cocoa, soft drinks such as and fruits juice, alcoholic beverage such as medicated liquor, snacks such as candy (drops), and chocolate, chewing gum, Japanese cracker, azuki-bean jelly and so forth, to produce the functional food or healthy food having preventing or treatment effect of periodontal disease.

Note that the compositions is added to yogurt, soy source, drinks and the like, solubilizing auxiliaries or the stabilizers may be employed not so as to form crystalline of the composition of the present invention to precipitated.

### EXAMPLES

The present invention is explained in detail by using examples below, however, the present invention is not limited to them.

### Example 1

### Effect of the Dental Root-Periodontal Tissue Formation

### (1-1) Diagnostics

Dulbecco′ phosphate buffered saline (hereinbelow, it is some times referred to as “D-PBS”) was purchased from Dainippon Pharmaceuticals Inc. Affi-Gel agarose beads (Affigel heparin Cat. No.: 15306173) were purchased from Bio-Rad Laboratories, Inc. Five days age of C57BL/6 mice were purchased from Sankyo Labo Service Corporation. As host mice, 10 to 15 weeks age of C57BL/6 mice (female) were purchased from Sankyo Labo Service Corporation, and used two per group. Since it was know that a kidney and an anterior chamber of eye hardly have immune response among organs in adult mice, it was not necessary to especially use nude mice in graft culture.

### (1-2) Preparation of Absorptive Beads Including Heparin Honokiol Impregnation

Honokiol-impregnated agarose beads were prepared as follows. Honokiol was diluted to the concentration of 10 μM by using D-PBS. The agarose beads were washed with D-PBS.

After washing, four agarose beads were placed in the 1.5 mL tube and 100 μL of the honokiol solution was added into it. Then, the tube was incubated at room temperature for 45 minutes to impregnated honokiol into the agarose beads. As mentioned above, absorptive beads including heparin honokiol impregnation (hereinbelow, it is sometimes referred to as “honokiol-impregnated agarose beads”) were prepared.

Alternatively, another four agarose beads were placed in the 1.5 mL tube and 100 μL of D-PBS was added into it to prepare D-PBS-impregnated agarose beads were prepared, and they were used as negative control.

### (1-3) Confirmation of Effect of the Dental Root-Periodontal Tissue Formation

(1-3-1) Embedding into Dental Pulp

Five days age of C45BL/6 mice, where their dental root-periodontal tissue was just before formed, were anesthetized and sacrificed by decapitation. After excised lower jaw of them, the first molars in the lower jaw were excised with dental follicles. One of the honokiol-impregnated agarose beads prepared in the (5-1) was embedded into the dental pulp of the excised teeth, the first molar in the lower jaw.

As the negative control, one of the D-PBS-impregnated agarose beads prepared was embedded into the dental pulp of another excised teeth, the first molar in the lower jaw.

### (1-3-2) Graft Culture

The excised teeth under which the honokiol-impregnated agarose bead was embedded, and that under which the D-PBS impregnated agarose bead was embedded, were respectively grafted under capsule of kidney of the host mice. The host mice were bred under the condition of 12 hours lighting, at room temperature, 25° C., free taking feed and water for three weeks.

After three weeks from grafting of the excised tooth, the host mice were sacrificed by dislocation of cervical vertebrae to excise the kidney to recover the cultured excised tooth (grafted tooth).

Formation of dental root-periodontal tissue (extension), coat of the dental root, and bone of the grafted tooth, which was recovered, were observed under stereoscopic microscope, and then subjected to tomography by using micro CT. Then, images were structured in three dimensions.

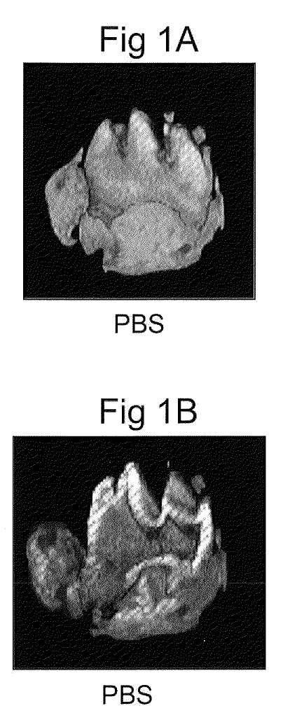

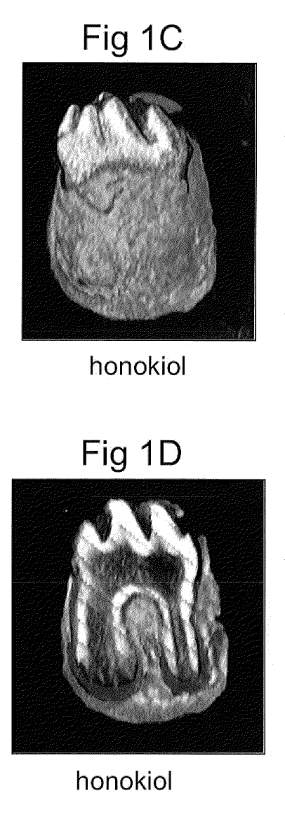

Observation results of the graft tooth after the graft culture were shown in FIG. 1. In FIG. 1, tooth formed were colored in white, the periodontal ligament were colored in black, and the alveolar bone was hatched.

From the result shown in FIG. 1, little extension of dental root and bone formation were observed in the negative control group, in which D-PBS was used (see FIGS. 1A and 1B, shown as “PBS”). In contrast, prominent extension of dental root compared to the negative control group were observed in the experiment group using the honokiol-impregnated agarose bead (see FIGS. 1C and 1D, shown as “honokiol”).

Furthermore, in all of the graft samples, under which the honokiol-impregnated agarose bead was embedded and the extension of dental root were observed, bone formation around the newly formed dental root were also observed. Therefore, these were pictured by using soft X-ray, and checked whether dental root were extended inside of the alveolar bone.

As a result, the extension of dental root was observed inside the grafted tooth inside of the formed bone (see FIG. 1D).

Furthermore, between the dental root and the bone formed, the structure similar to periodontal ligamentous band was observed. Note that the newly formed alveolar bone was shown as a hatched part in FIG. 1. The parts with no hatch of “side” and “whole” in three dimension structured images, and a white part without hatch show in “section” showed tooth. Furthermore, in the “section” image in FIG. 1C, a black part showed periodontal ligament.

According to these results, it was demonstrated that the pharmaceutical preparation comprising honokiol as the active ingredient had excellent promotion effect for dental root-periodontal tissue formation (extension), and it also had promotion effect for surrounding tissue such as the periodontal ligament and alveolar bone, which support the formed dental root.

Therefore, it was demonstrated that the pharmaceutical preparation comprising honokiol as the active ingredient was very useful for promoting dental root, and surrounding tissue such as periodontal ligament or alveolar bone tissue to support the newly formed dental root.

Accordingly, the pharmaceutical preparation for preventing and/or treating periodontal disease has possibilities for the wide application range, in a variety of scene wherein maintenance of periodontal tissue or promotion to form the tissue. For example, it is considered to be particularly available in the following ways.

(1) In dental field, dental root-periodontal tissue formation may be promoted by adding the agent to the tooth paste or a gargle.
(2) As general foods, gum or candies having preventing and/or treating periodontal disease may be prepared by adding the agent in a small amount.
(3) As animal companion related goods, pet food or beverages having preventing and/or treating periodontal disease may be prepared by adding the agent in a small amount.

### INDUSTRIAL APPLICABILITY

The present invention of the pharmaceutical composition, the pharmaceutical preparation, health food, and the functional food comprising the compound shown in the above formula (I) as the active ingredient is applicability in each field.

1. A pharmaceutical composition for preventing and/or treating periodontal disease comprising at least one having dental root-periodontal tissue formation promotion effect selected from the group consisting of a compound shown in a following formula (I), a physiologically acceptable salt thereof, and a hydrate thereof. 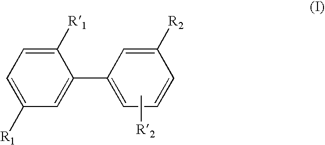
(In the formula, R₁ and R₂ are independently functional groups selected from a group consisting of a substituted or non-substituted linear alkyl group, branched alkyl group, linear alkenyl group and branched alkenyl group with having the carbon number of 3 to 5. R′₁ and R′₂ are independently a hydrogen atom, hydroxyl group, or alkoxy group having the carbon number of 1 to 3.)

2. The pharmaceutical composition according to claim 1, wherein the compound shown in the above-mentioned formula (I) is one having dental root-periodontal tissue formation promotion effect selected from the group consisting of that shown in the following formula (II), (III), the physiologically acceptable salts thereof, and hydrate thereof. 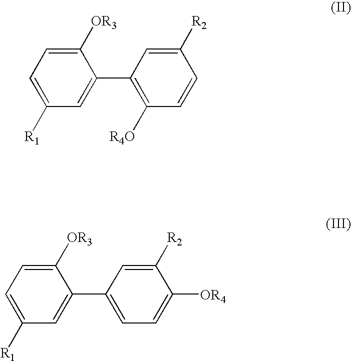
(In the formula, R₁ and R₂ are independently functional groups selected from a group consisting of a substituted or non-substituted linear alkyl group, branched alkyl group, linear alkenyl group and branched alkenyl group with having the carbon number of 3 to 5. R₃ and R₄ are independently a hydrogen atom, hydroxyl group, or alkoxy group having the carbon number of 1 to 3.)

3. The pharmaceutical composition according to claim 2, wherein the compound shown in the above-mentioned formula (I) is one having dental root-periodontal tissue formation promotion effect selected from the group consisting of that shown in the following formulae (IV), (V).

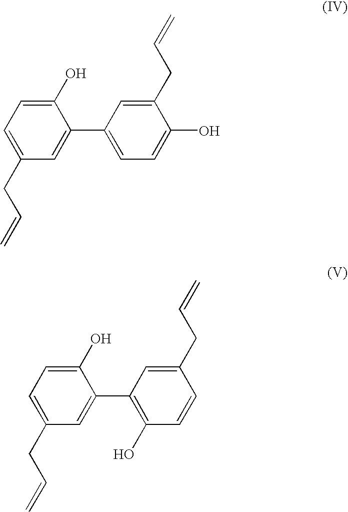

4. The pharmaceutical composition according to one of claim 1 to 3, wherein one selected from the group consisting of: the compound shown in the following formulae (I) to (V); the physiologically acceptable salt thereof; and the hydrate thereof; is obtained by extraction from a plant selected from the group consisting of Magnolia officinalis, Magnolia officinalis var. biloba, Magnolia hypoleuca, Magnolia macrophylla, Magnolia obovata, Magnolia salicifola, Magnolia stellata, Magnolia virginiana, Magnolia delavayi, Magnolia kobus, Magnolia sieboldii, and Magnolia wilsonii, having the dental root-periodontal tissue formation promotion effect.

5. A pharmaceutical preparation for preventing and/or treating periodontal disease comprising at least one selected from the group consisting of the compound, the physiologically acceptable salt thereof, and hydrate thereof according to the claim 1.

6. The pharmaceutical preparation for preventing and/or treating periodontal disease according to the claim 5, wherein one selected from the group consisting of the compound, the pharmaceutically acceptable salt thereof, and hydrate thereof according to the claim 1 is obtained by extraction from the plant selected from the group consisting of Magnolia officinalis, Magnolia officinalis var. biloba, Magnolia hypoleuca, Magnolia macrophylla, Magnolia obovata, Magnolia salicifola, Magnolia stellata, Magnolia virginiana, Magnolia delavayi, Magnolia kobus, Magnolia sieboldii, and Magnolia wilsonii, having the dental root-periodontal tissue formation promotion effect.

7. A functional food comprising one selected from the group consisting of the compound, physiologically acceptable thereof, and the hydrate thereof according to the claim 1.

8. The functional food according to the claim 7, wherein one selected from the group consisting of the compound, physiologically acceptable thereof, and the hydrate thereof according to the claim 1 is extracted from the plant selected from the group consisting of Magnolia officinalis, Magnolia officinalis var. biloba, Magnolia hypoleuca, Magnolia macrophylla, Magnolia obovata, Magnolia salicifola, Magnolia stellata, Magnolia virginiana, Magnolia delavayi, Magnolia kobus, Magnolia sieboldii, and Magnolia wilsonii, having the dental root-periodontal tissue formation promotion effect.

9. A healthy food comprising one selected from the group consisting of the compound, the physiologically acceptable salts thereof, and the hydrate thereof according to the claim 1, having the dental root-periodontal tissue formation promotion effect.

10. The healthy food according to the claim 9, wherein one selected from the group consisting of the compound, the physiologically acceptable salts thereof, and the hydrate thereof according to the claim 1 is obtained by extraction from the plant selected from the group consisting of Magnolia officinalis, Magnolia officinalis var. biloba, Magnolia hypoleuca, Magnolia macrophylla, Magnolia obovata, Magnolia salicifola, Magnolia stellata, Magnolia virginiana, Magnolia delavayi, Magnolia kobus, Magnolia sieboldii, and Magnolia wilsonii, having the dental root-periodontal tissue formation promotion effect.
